(19) United States
(12) Patent Application Publication
(10) Pub. No.: US 2007/0048731 A1
(43) Pub. Date: Mar. 1, 2007

(54) High throughput use-dependent assay based on stimulation of cells on a silicon surface

(75) Inventors: Michael A. Colicos, Calgary (CA); Gerald Zamponi, Cochrane (CA)

Correspondence Address: FISH & RICHARDSON P.C., PO BOX 1022, MINNEAPOLIS, MN, 55440-1022, US

(73) Assignee: Neurosilicon, Calgary (CA)

(21) Appl. No.: 11/439,377

(22) Filed: May 22, 2006

Related U.S. Application Data
(60) Provisional application No. 60/683,132, filed on May 20, 2005.

(51) Int. Cl. C12Q 1/00; C12M 3/00

(52) U.S. Cl. 435/4; 435/287.2

(57) ABSTRACT

The invention pertains generally to the field of drug discovery science. More specifically, the invention refers to a drug screening assay and device that is used to test the effect of specific chemical compounds as use dependent ion channel blockers. The invention includes a unique method for stimulating cells with photoconductive stimulation and reading consequent cell ion channel activity with the optical imaging of fluorescent dyes.

### CROSS REFERENCE TO RELATED APPLICATIONS

This application claims the benefit of priority under 35 U.S.C. § 119 to U.S. provisional application, having Ser. No. 60/683,132 filed May 20, 2005, the disclosure of which is incorporated herein by reference in its entirety. The disclosures of U.S. provisional applications having Ser. Nos. 60/689,645 filed Jun. 10, 2005, 60/691,012, filed Jun. 15, 2005, 60/691,322, filed Jun. 15, 2005, and 60/699,829, filed Jul. 15, 2005 are incorporated herein by reference in their entirety.

### FIELD OF THE INVENTION

This invention relates to the field of drug discovery science, and more particularly to methods and devices for screening compounds based on their effect on repetitively stimulated cells on a photoconducting silicon surface.

### BACKGROUND

Certain physiological processes rely on cells that are excitable. A cell is excitable if it generates an action potential, which is a rapid and dramatic change in the cell's electrochemical potential. In particular, an excitable cell has a membrane potential because the inside and outside surfaces of its membrane carry different electrical charges. If the potential difference is destroyed—a process known as depolarization—the cell will ultimately die. Ion channels in the cell membrane play a key role in the electrophysiological properties of excitable cells. An intake or exit of a quantity of ions—e.g., cations of metals such as sodium, calcium, and potassium, and anions of elements such as chlorine—through a cell's ion channels typically acts as a switch that controls the cell's behavior. Consequently, drugs that block or activate ion channels are used to treat a number of diseases and conditions.

However, in many instances, an ion channel behaves in a use-dependent manner. Opening a channel once constitutes a single event, but multiple repetitive uses of the channel change the channel's characteristics. Such multiple uses of a channel are said to wear the channel down. In order to replicate physiological behavior, it is necessary to wear a channel down by repetitive stimulation before a drug candidate's action against it can be tested.

Controllable manipulation of cellular excitation is central to investigating the effect of drug candidates on physiological processes and functions of excitable tissues. In many instances it is necessary to be able to achieve stimulation of the cells multiple times instead of just once, e.g., because a neuron is only triggered after it has received sufficient stimulation. Frequently, patch clamp techniques have been used to monitor the ionic basis of cellular behavior. In cell-attached patch-clamping, a micro-capillary pipette is placed in contact with an area of a cell membrane that includes an ion channel, and conductivity measurements are made over that region alone. Although effective, this approach allows the analysis of only one region of one cell at any given time and requires significant skilled human labor.

Accordingly, it is more useful to try to monitor behaviors of whole cells singly or in groups, for example in the case of neurons, networks of neurons. The issues involved are mainly: developing ways of stimulating an activity of interest in the cell(s); achieving the stimulation selectively, on a cell-by-cell basis; reliably monitoring the activity and its change in response to an external stimulus, such as increase in concentration of a drug molecule; and ensuring that the stimulation can be achieved repetitively.

A variety of electrical and chemical methods for cell stimulation has been devised over the years. The earliest approaches involved stimulation with electrodes, or chemical modulation of voltage-gated and ligand-gated ion channels. More recently, interfaces between silicon technology and manipulation of living cells have opened new techniques for achieving non-invasive extracellular stimulation. When combined with model systems such as dissociated neuronal cultures or organotypic preparations, a silicon interface provides a powerful tool for examining neuronal network behavior (see, e.g., Pine, J., “Recording action potentials from cultured neurons with extracellular microcircuit electrodes,”J. Neurosci. Methods, 2, 19-31, (1980); Gross, G. W., Rhoades, B. K., Azzazy, H. M. E., and Wu, M. C., “The use of neuronal networks on multielectrode arrays as biosensors,” Biosen. Bioelec., 10, 553-567, (1995); Maher, M. P., Pine, J., Wright, J. and Tai, Y. C., “The neurochip: a new multielectrode device for stimulating and recording from cultured neurons,” J. Neurosci. Methods, 87, 45-56, (1999); Kaul, R. A., Syed, N. I., and Fromherz, P., “Neuron-semiconductor chip with chemical synapse between identified neurons” Phys. Rev. Lett., 92, 038102, (2004), all of which are incorporated herein by reference in their entirety). For example, an array of transistor interfaces has been used to stimulate and acquire detailed measurements of membrane potential changes of neurons positioned over a given transistor element. However, the utility of the transistor array is constrained by the fixed spatial position of the transistor elements; the positions of individual neurons in a neuronal network cannot be guaranteed to align perfectly with the transistor elements and thus the individual neurons cannot always be selectively stimulated.

A number of optical methods for eliciting neuronal excitation have also been described. For instance, cell-specific expression of genetically encoded light-sensitive controllers of membrane voltage can be used to manipulate cell excitability (reviewed in, e.g., Miesenbock, G., Kevrekidis, I. G., “Optical imaging and control of genetically designated neurons in functioning circuits,” Ann. Rev. Neurosci., 28, 533-63, (2005), incorporated herein by reference). In addition, neurons can be incubated either chronically or acutely in solutions containing caged ions, or caged neurotransmitters. These caged ions or neurotransmitters cannot act upon ion channels because they are bound to a carrier molecule that prevents their activity. The carrier molecule can be unbound from the ion or neurotransmitter using a light source such as a laser. Lasers can be used to evoke synaptic responses, for example, by uncaging either the extracellular neurotransmitters or intracellular calcium at nerve terminals to promote synaptic release (see, e.g., Denk, W., Delaney, K. R., Gelperin, A., Kleinfeld, D., Strowbridge, B. W., Tank, D. W., and Yuste, R., “Biological Anatomical and functional imaging of neurons using 2-photon laser scanning microscopy,” J. Neurosci. Methods, 54, 151-162, (1994); Callaway, E. M., and Yuste, R., “Stimulating neurons with light,” Curr. Opin. Neurobiol., 12, 587-592, (2002), both of which are incorporated herein by reference in their entirety). These techniques, although offering the possibility of repetitive stimulation of cells, suffer from the drawback that the cells must be pre-incubated with a special compound that releases the neurotransmitter when excited by a laser. These techniques are therefore invasive. Uncaging complicates interpreting the results of these measurements because the neurotransmitter diffuses away from its normal synaptic localization. Furthermore, there are limitations on the duration of stimulation that is possible.

Recently, a light addressable technique for stimulation of targeted neurons that is based on photoconducting properties of silicon has been developed (see, Colicos, M. A., Collins, B. E., Sailor, M. J., and Goda, Y., “Remodeling of synaptic actin induced by photoconductive stimulation,” Cell, 107, 605-616, (2001), incorporated herein by reference). A light shined on a selected area of a silicon wafer generates a photocurrent in that area when a voltage is applied to the silicon. By holding the light constant and pulsing the voltage, repetitive stimulation of a cell in the selected area can be achieved. This method thereby interfaces a complex neural network, such as formed from a group of neurons, with a method that harnesses true random-access capability. In contrast with other techniques described herein which have been mainly used in a research capacity for investigating short-term investigations of basic neuronal function, the photoconducting protocol is unique amongst light-directed cell excitation methods in offering non-invasive repetitive stimulation of cells. It is also straightforward to implement and is highly cost-effective. It permits spatially selective excitation of an area within 100 μm², a resolution that is not easily achievable with other methods of extracellular field stimulation. However, hitherto it has only been used as a research tool for investigating structural changes that result from long-term cellular excitation, for example, in elucidating how long-term memories are established by neuronal networks.

The photoconductive stimulation protocol has allowed the patterned stimulation of neural networks (X. Y. Tang, R. C. Gerkin, X. L. Wu, Y. Goda, and G. Q. Bi, “Light-directed, patterned stimulation of neuronal networks on silicon chips”, SFN Annual Meeting, Program No. 920.4, (2004)) and the study of activity-dependent synapse formation by observing the remodeling of synaptic cytoskeleton as a result of stimulation (Colicos, M. A., et al., Cell, 107, 605-616, (2001)). The photoconductive stimulation system has also been modified to integrate a laser beam and an acousto-optic deflector, with which a complex spatiotemporal stimulation pattern can be generated to study detailed network properties (Starovoytov, A., Choi, J., Seung, H. S., “Light-directed electrical stimulation of neurons cultured on silicon wafers,” J. Neurophysiol., 93(2), 1090-8, (2005)). In this method, a constant voltage is applied to a silicon substrate, and cells are entrained using a light source pulsed on to selected areas. However, to date, photoconductive stimulation of cells has not formed the basis of a screening assay.

To discover drug candidates efficiently, high throughput assays are typically used. Such assays facilitate the process of testing many candidate molecules on an intended target. Liquid handling robotic systems rapidly apply test compounds to multi-well plates and permit a given assay to be run in multiplex fashion. There are primarily two types of assays that are used: the radioligand binding assay, and the fluorescent based screening assay. However, hitherto these two types do not allow assaying of drugs that require repetitive stimulation of ion channel activity in order to reach their maximum effects because an underlying way of repetitively stimulating cells has not been available in the respective assay format.

In a radioligand binding assay, the ability of a given compound to displace a radioactive molecule from an intended target is measured biochemically. The drawback of this assay in the context of ion channel activity is that it is a static test that is not based on the measurement of a drug's effects on ion channel activity per se. In other words, even if a drug binds to the channel, the drug may not be biologically active because the cell may not have been appropriately stimulated. This invalidates many of the findings from a radioligand binding assay.

The typical fluorescent based assay device is made up of a multi-well plate that allows a specific compound to be added to a group of cells. In a typical screening run to test compounds with ion channel activity, a compound is added to a well along with a fluorescent dye. The dye that is used depends on which ion channel is to be analyzed. The dye interacts with the ion, resulting in a change in the fluorescence from the dye, which can be measured optically. This reflects the global concentration of the ion within each cell. The effect of the compound on ion influx is then measured by a change in fluorescent intensity of the dye.

To cause cellular stimulation and ion channel activity in fluorescent based screening for drugs, cells are loaded with a fluorescent dye and depolarized with, e.g., an electrolyte such as potassium chloride, thereby stimulating cells one time but in a highly controlled manner. Various compounds are applied to the cells and assessed for their abilities to block this ion channel activity through subsequent stimulation. One major drawback of this type of assay is that each cell is depolarized only once, by immersion in, e.g., KCl, and for a prolonged time, thus rendering the culture incapable of subsequent screening through subsequent stimulation because the KCl cannot be removed. Another major drawback of this type of assay is that prolonged depolarization causes excito-toxicity in neuronal cells that arises when a cell is overstimulated or depolarized for too long, triggering apoptotic processes and causing the cell to die. Neuronal excito-toxicity and apoptotic processes might introduce confounding factors into these types of experiments and lead to flawed results. This technique, with the aforementioned limitations, does not allow for the screening of compounds that inhibit ion channels by a mechanism called use-dependent block. Many compounds only interact with ion channels after the ion channels have opened, or when they are in a deactivated state. This deactivated state occurs most often after a channel has opened for a certain period of time, i.e., after it has been repeatedly stimulated. Compounds that act as use-dependent blockers increase their effectiveness with increased electrical cellular activity. For example, a number of antiepileptic drugs and anti-arrhythmic drugs function as use dependent blockers. These drugs frequently act only on channels that are either open or deactivated, which usually requires repetitive stimulation. The conventional fluorescent based and radioligand assays do not allow for repetitive cell stimulation, but rather stimulate the cell only once. Thus, many potentially useful drugs cannot be identified by either of these conventional screening processes.

Patch clamping is also not practical for testing potential drug candidates. In other words, the patch clamp system can stimulate cells repetitively but it cannot do this in a highly parallel and automated fashion.

Therefore, one of the limiting factors in current drug screening technology is the inability to find therapeutically active frequency-dependent ion channel inhibitors easily and quickly using automated testing systems. Hitherto, assays using such systems are either limited to stimulating cells only once or are not able to selectively stimulate particular cells. Consequently, although currently available robotic screening devices can test many compounds quickly and automatically they cannot stimulate cells repetitively.

Hitherto, the photoconductive stimulation method has not been adapted for high throughput screening. There therefore exists a need in the art for a method to selectively stimulate cells repetitively and to test many compounds automatically and concurrently.

The discussion of the background to the invention herein is included to explain the context of the invention. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY OF THE INVENTION

In one aspect, the invention provides an assay system which allows for repetitive cell stimulation and high throughput screening of test compounds, the system comprising: a silicon wafer having a surface suitable for cellular growth, and configured to be in contact with a growth medium, wherein the growth medium contains the test compound; at least one cell in contact with the surface; a light source configured to direct a light pulse to repetitively stimulate the at least one cell; control circuitry connected to the silicon wafer and to the light source; and a detector configured to detect the biochemical effects of the test compound on the at least one cell.

The invention also provides a method for measuring the effect of a test compound on cellular activity, the method comprising: providing a silicon wafer having a surface suitable for cell growth; culturing at least one cell on the surface; contacting the at least one cell with a test compound; repetitively stimulating the at least one cell so as to excite the cell by directing a light pulse onto the at least one cell, wherein the light pulse is controlled by control circuitry; and detecting a biochemical effect of the test compound on the at least one cell as a result of the stimulated excitation of the cells.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic cross-sectional view of a well in one embodiment of the invention with the following features:

FIG. 2: Depolarization of human embryo kidney (HEK) cells expressing calcium channel subunits.

FIG. 3: Bottom plate containing photoconductive stimulation light source. Top view and cross-sectional view (bottom).

FIG. 4: Bottom portion of middle plate containing sockets for wafer plus bottom electrode. Top view and cross-sectional view (bottom).

FIG. 5: Individual wafers arranged in a 8×12 array. Top view and cross-sectional view (bottom).

FIG. 6: Top portion of middle plate containing wells and upper electrodes. Top view and cross-sectional view (bottom).

FIG. 7: Top plate containing imagers and associated dichroic mirror and emission and excitation filters. Top view and cross-sectional view (bottom).

FIGS. 8A and 8B: Cross-sectional view of all plates. The upper set is the exploded view, while the lower set represents the plates in the closed position.

FIG. 9: One example of the dichroic mirror and associated emission and excitation filters. Upper diagram is top view, while lower diagram is the side view.

### DETAILED DESCRIPTION

The present invention pertains generally to the field of drug discovery. More specifically, the invention relates to drug screening assays and devices that are used to test the effect of specific chemical compounds as use-dependent ion channel blockers. In one aspect, the invention includes a method for exciting cells with photoconductive stimulation, and reading consequent cell channel activity by optical imaging of fluorescent dyes.

Photoconductive stimulation allows the non-invasive depolarization of neurons cultured on a silicon wafer. The method uses a beam of light to target a cell or cells of interest while applying a voltage bias across the silicon wafer. A targeted cell is excited with minimal physiological manipulation. Although functionally similar to transistor based neuronal interfaces, the photoconductive stimulation method has the advantage of being able to excite any neuron in a network regardless of its spatial position on the silicon substrate. The use of opto/electronics, as opposed to chemicals, as the stimulation system, minimizes the risks of interference by the stimulator on normal cellular activity. Furthermore, using a programmable light-addressed/electric field pulse allows for the stimulation of cells in a pre-defined pattern to simulate specific cellular activity. This protocol can be implemented on a conventional reflected light fluorescence microscope using materials and resources that are readily available. When combined with fluorescence imaging of various molecular probes, activity-dependent cellular processes can be dynamically monitored.

The present invention provides a method and device that repetitively stimulates cells without adversely affecting them and allows for fluorescent based measuring methods to be used to identify compounds that can act on ion channels in a use dependent manner.

In the present invention, cells are excited repetitively through photoconductive stimulation. This method can induce rapid activity in cells, e.g., human embryo kidney cells, neuronal cells, and heart cells. This repetitive stimulation more closely reflects the physiological behavior of ion channels in cells in comparison to other techniques in the art which stimulate the cells once only.

The present invention provides a novel drug screening assay and device which successfully permits high throughput screening of compounds that affect ion channels in a use-dependent manner. The invention does so by exciting cellular activity by photoconductive stimulation and by testing multiple compounds in parallel.

Prior to the instant invention, there was no method available to selectively stimulate cells repetitively and to test many compounds automatically and concurrently. This invention can be deployed by many different high throughput systems, including liquid handling robotic systems.

Thus, the instant invention saves significant costs in the drug screening process, particularly as applied to searching for drug candidates that are capable of exciting cells, blocking excitation of cells, blocking ion channels, preventing ion channels from re-entering an active state, or preventing ion channels from being deactivated.

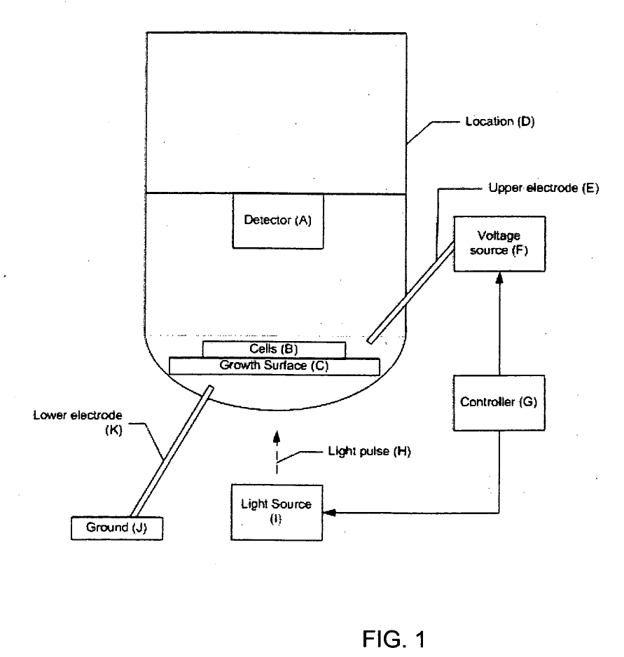

A portion of a device according to the invention is shown schematically in FIG. 1, wherein a detector system (A) detects the effects of a test compound on cells (B) that are to be stimulated and analyzed. The growth surface (C) upon which the cells are grown is situated within a location (D), in which the test compound will be tested. A control circuit comprises an upper electrode (E), a power/voltage source (e.g., a battery) (F) which delivers power to the location, a ground source (J), a lower electrode (K) to ground the current, and a controller (G), such as a microcomputer, that controls the power and light sources and the assay process in general. Light source (I) directs a light pulse (H) towards the cells from underneath the silicon wafer. The light beam travels through the wafer and causes a photocurrent on the opposite side of the wafer in the immediate vicinity of the cells.

An array of independently controllable systems such as those of FIG. 1, in which each of the systems is located in a well of an assay plate is preferably used for high throughput screening.

Assay Plate

As used herein, the term “assay plate” means a piece of apparatus having multiple locations on which, or in which, samples can be retained, while measurements on the samples are made. In preferred embodiments, the apparatus is a tray or a plate and the locations are wells. Even more preferably, the assay plate has 96 locations.

One embodiment of devices according to the instant invention comprises a plate with a definable number of wells, preferably 96 wells. Preferably the plate is a standard assay plate, as used in conjunction with automated or robotic devices used in various screening technologies. The assay plate may be made of plastic or other suitable material. The surface upon which cells are grown may be a silicon wafer (sometimes referred to as a silicon die) that is placed in each well.

Screening

As used herein, the term “high throughput screening” means an automated methodology to screen multiple compounds simultaneously and in succession.

Overview

In a preferred embodiment, within each well of an assay plate, a silicon wafer is mounted so that a light source can be directed to the lower surface of the wafer, and such that the target cells are grown on the upper surface. The cultured cells are not required to be positioned in any specific geometry on the silicon. A substrate which promotes cellular growth and survival on silicon is used. Optical techniques based on the detection of changes in fluorescence intensity are preferably used to measure the response of the cells to external stimuli such as presence of a concentration of a test compound. The device uses cells grown on silicon wafers, targets specific subset groups of the cells in each well using coherent light, and, by the application of an electric field, causes their specific and repetitive stimulation. Useful cells include human embryo kidney cells, neuronal or cardiac cells that endogenously express ion channels, as well as cells that express cloned, exogenously introduced ion channels. A subset of cells, which may be a subset of the cultured cells on a given wafer, is targeted with a light beam, which creates a high conductance path through the wafer underneath the targeted cells, and subsequently stimulates those cells specifically with a voltage pulse applied across the wafer and cell culture. Traditional ion sensitive fluorescent dyes are loaded into the cells prior to the assay to measure changes in ion channel activity and to track, for example, calcium or sodium entry into the cell.

Repetitive stimulation preferably occurs over a period of 1-30 seconds, even more preferably over a period of 5-25 seconds, still more preferably over a period of 10-20 seconds, and yet more preferably over a period of about 15 seconds, or 15 seconds. The assay measurements may take on the order of several minutes, for example, two minutes, and several separate periods of repetitive stimulation may occur during a given assay.

Exemplary Device

One embodiment of the invention is a device that uses cells grown on silicon wafers located in wells on an assay plate, targets specific subsets of the cells in each well using coherent light, and, by the application of an electric field, causes their specific and repetitive stimulation.

The stimulation causes the rapid, periodic, reproducible and controlled excitation of the cells for a predefined period, usually a few milliseconds. Test compounds are added to the plate of wells to investigate their effect on the ion channels of the stimulated cells within. The intensity levels of fluorescent dyes are optically measured and used to assay the effect of the drugs on the ion channels.

Systems of the present invention for stimulating neurons may comprise: a silicon wafer that acts as a substrate that promotes cellular growth and survival; a light source for sending a light pulse to target a specific subset of neurons; a set of electrodes to stimulate the specifically targeted cells; the assembly of multiple devices in a plate of wells, with individual cell lines or native cells grown on the silicon wafers in each well; an upper light source to stimulate the fluorescent dye in the cells; a photodetector system to differentially read the light intensity from both stimulated and non-stimulated cells; and a microchip to control the electric pulse and targeting light source in specific patterns. The lower, targeting, light source is configured to generate a specific frequency of light. The plate may be constructed of plastic with a user definable number of wells.

Each well plate preferably comprises a hardwired connection to a controller, which may be a microcomputer. The microcomputer may comprise a processor, a connection to a power source and a wired connection to each plate well. The wired connection to each well may include an electrical connection across the silicon wafer. The microcomputer may comprise a wired or wireless connection to the targeting light source.

Test Compounds

As used herein, the term “test compound” includes any molecule that is suspected to have a biological effect such as action in a cell. A test compound may be a small organic molecule having, say, 50 or fewer non-hydrogen atoms, or a peptide, an oligopeptide, a nucleotide, an oligonucleotide, or a protein, typically a small protein having a molecular weight <5,000 Daltons.

Dye

When using fluorescent detection, a fluorescent dye added to the growth medium. As used herein, the term “fluorescent dye” means a molecule that emits light when stimulated by one or more incident photons. The light is emitted when the molecule transitions from an excited state to a ground state. The excited state is preferably a singlet-state, when the ground state is also a singlet state. The molecule is preferably a small organic molecule, but may also be a protein. Many molecules having a fluorophore are known in the art.

A fluorescent dye may be loaded onto the cell lines or native cells at each location, and then the cells are exposed to a test compound. For example, a solution of test compound may be added to the cells in each well. The change in fluorescence in each well of the stimulated cells is measured by comparison to un-stimulated cells by means of a detector. A specific frequency of light may be delivered to the cells to excite the particular fluorescent dye being used. The emitted light from the fluorescent dye is filtered out and detected by the photodetection system. The photodetection system can be alternated between the region where the drug has been added and the region where no drug has been added.

Wafer

The silicon wafers used with the present invention are preferably p-type, boron-doped, having a resistivity of 10-20 ohm-cm, an exposed (100) lattice face, and are single-sided polished, between 0.05 and 0.7 mm thick, preferably between 0.3 and 0.5 mm thick, for example 0.4 mm thick. It is important to use a (100)-oriented crystal of silicon because, in that orientation, a beam of light directed onto the surface will travel through the body of the silicon along a cylindrical corridor without significant lateral dispersion. That way, a silicon wafer can be illuminated on a face opposite to the face on which cells are cultured, and still ensure stimulation of the cells.

Preferably, the cut wafers are then sterilized, e.g., with absolute alcohol.

The assay plates are then allowed to sit overnight in a buffered solution of poly-D-lysine. A preferred buffer is borate. The wells are then optionally coated in laminin. Where applicable, laminin application is preferably coordinated with the cell preparation to allow treatment of the wafers for 3-4 hours prior to cell plating. Altering this time can result in altered morphology or growth characteristics of the neurons. In the case where the cells are human embryo kidney cells, laminin is not found necessary.

Laminin, if used, must be thoroughly washed from both sides of the wafers, preferably with 3 washes of phosphate buffered saline (PBS), with no delay between solution changes. This should be done immediately prior to plating the cells.

Any cell culture preparation protocol that produces viable cultures on glass slides can be used with silicon wafers prepared as described hereinabove.

Cell plating is achieved by incubating a suspension of the cells, diluted to give an appropriate cell plating density, with the silicon wafers. The incubation medium can be washed and replenished as necessary. Preferred cell plating densities are in the range 10³-10⁵ cells per cm² of wafer. Even more preferably the plating density is 10⁴ cells per cm².

Operation of Assay

During operation, each light pulse incident on the wafer causes an increase in conductivity of the silicon wafer at the region of incidence, below the specific targeted cells. This increase is due to the generation of large numbers of electron-hole pairs, created by the energy of the light pulse. This is also termed the photoconductive effect. By applying a voltage pulse across the entire circuit that includes the silicon wafers, an electric charge is transferred to the targeted cells, thereby resulting in their depolarization. With a rapid sequence of light or current pulses, the cells continually fire as long as the opto-electronic stimulation persists.

Compounds to be tested are then added to individual wells. This process can be automated by liquid handling robotic systems as typically used for drug screening. Such devices include those that utilize robotic arms, and may include a pipette system to add test compounds to the cell surface.

At the time of the assay, the lower light source will be activated to target a sub-region of the wafer, the timed pulses of electric stimulation will be applied by a voltage from the platinum electrode to the base contact of the silicon wafer (or the bottom electrode). The base contact may consist of a transparent conductive material coating the bottom surface of the wafer, electrically connected by a grid of wire contacts. The frequency and pattern of stimulation is preferably programmable through an interface mounted in the base of the well plate. Timing of the stimulation is preferably coordinated with illumination and measurement with photomultipliers, e.g., of the fluorescent dyes from the circuitry mounted in the lid of the well plate. Therefore a test compound's effect on the targeted ion channel during repetitive stimulation is measured indirectly by its ability to modulate the fluorescent signal.

When a cell is described as being in contact with the silicon surface, it is understood that the better the contact, the better the level of depolarization of the cells upon photoconductive stimulation of the silicon surface. HEK cells form especially good contact with the surface without requiring any assistive structure. Neurons, on the other hand, perform more effectively with a biochemical support layer, such as a layer of glial cells, or other cells that provide nutrients that allow the neurons to survive.

A principal application of the photoconductive stimulation device as described here is for the acute, non-invasive stimulation of neurons from dissociated hippocampal co-cultures. Successful depolarization of the cells will elicit an action potential, which can be detected by either optical imaging of a fluorescent calcium indicator or voltage sensitive dye, or by direct electrophysiological recording. Video imaging of calcium in hippocampal neuron stimulated at 1 Hz can be viewed in the Supplementary data of Colicos, et al., 2001 (incorporated herein by reference in its entirety). In addition to hippocampal neurons, cortical neurons and retinal ganglion cells have also been successfully cultured on silicon wafers, depolarized and fired using the methods described herein. Additionally, HEK cells transfected with voltage sensitive calcium channel subunits can be depolarized, as further described herein. Therefore, many excitable cell types amenable to electrical depolarization can be stimulated using this device.

Another application of the device as described herein is for live cell video imaging of fluorescently tagged molecules, such as proteins fused to green fluorescent protein (GFP), and determining their response to activity. Using adequate perfusion equipment, cell cultures can be maintained and observed for hours, allowing the investigation of phenomena that occur over a longer time scale such as synaptic remodeling.

Photoconductive stimulation of excitable cells grown on silicon wafers is a protocol that provides core functionality for a variety of possible applications and devices. For example, simultaneous, multiple cell targeting systems, as well as environmentally regulated, extended stimulation devices are envisaged.

Light/Voltage Source(s)

As used herein, the term “light source” means a source of coherent light, such as a semiconductor laser, or a laser emitting diode, or a dye laser. In a preferred embodiment, the light source is kept constant and the voltage is varied. In another embodiment, the light source is varied, either by pulsing, or by varying its position of incidence on the wafer, while the voltage is kept constant. In some embodiments, the light source and voltage source are varied simultaneously. The light source directs the light pulse in a defined pattern at a defined wavelength and the voltage source directs the voltage pulse at the same time. The controller which controls the light source and voltage source may be a microcomputer.

The light beam is preferably incident on a region of silicon surface about 80-120 μm in diameter, and preferably about 100 μm in diameter. It is also consistent with the present invention that the region is 100-500 μm in diameter.

The light source for targeting the region of the wafer to be stimulated may be provided by an array of semiconductor lasers (alternately, light emitting diodes may be used, potentially with some optics), depending on the physiology of the specific cell type under investigation. Power and light intensity are based upon known parameters for photoconductive stimulation (see Colicos, et al., Cell, 107(5): 605-616, (2001), incorporated herein by reference). A pulse duration of a light source is typically in the range 0.1-10 ms, preferably 0.5-5 ms, even more preferably 1-2 ms, and yet more preferably about 2 ms.

More specifically, a subset group of cells in each well is targeted with a light beam, which creates a high conductance path through the wafer underneath the targeted cells, and subsequently stimulates those cells specifically with a voltage pulse applied across the wafer and cell culture. Traditional ion sensitive fluorescent dyes are loaded into the cells prior to the experiment to measure changes in ion channel activity and to track, for example, calcium or sodium entry into the cell.

The light source is preferably directed over a defined pattern, and at a defined wavelength. The wavelength will depend upon principally the fluorophore of the dye to be detected. Longer wavelengths, i.e., towards the red end of the visible spectrum are preferred. The light must be of the frequencies that are capable of producing a photocurrent in a silicon surface.

As used herein, the term “voltage source” means a source of electromotive force, such as a battery, or an electrolytic cell that is able to supply a voltage of between 3 and 8 V.

Any pulse generator capable of producing a 2 ms pulse of 0 to +5 volts can be used to supply the wafer with the desired frequency. Alternatively, more complex stimulation patterns can be achieved by using computer controlled electronics. Transistor-to-transistor (TTL) circuits typically have insufficient power to drive a device of the present invention, and therefore are preferably used only to trigger a power source.

Detector

As used herein, the term “detector” means a detecting system configured to measure the intensity of light from a fluorescent compound. The detector may comprise a combination of one or more ultraviolet light sources, one or more charged-coupled device (CCD) cameras, and one or more filters. In another embodiment, the detector may comprise an illuminating light source and a differentiating photodetector system. The photodetector system may be comprised of a single CCD or Complementary Metal Oxide Semiconductor (CMOS) based sensor per location, mounted in the light-sealed lid of the device.

In an embodiment of the invention, the detector detects fluorescence. In another embodiment one or more optical filters are positioned between the silicon wafer and the detector. The optical filters are used to distinguish between the light that excites the cells and the light that is emitted from the fluorescent dye. Each filter represents a different wavelength that is allowed to pass through the filter. The filter unit consists of two filters and a dichroic mirror. The dichroic mirror allows for one wavelength to proceed in one direction and another wavelength to proceed in a perpendicular direction. It is placed such that light is filtered before it reaches the photodetection device. One filter is responsible for letting the light that excites the cells from the light source to the surface and the other filter is used to allow just the fluorescence light from the surface to the photodetection system.

In another embodiment photoconductive stimulation may be used in conjunction with a radioligand binding assay.

Cells

The cells used with the present invention are any cells which express a voltage gated ion channel. Preferred cells are mammalian cells, and even more preferably human embryo kidney cells, neurons, cardiac cells, or any excitable cell that expresses an ion channel target. The present invention is also applicable to studying excitable behavior of non-neuronal cell types such as muscle cells and secretory cells. In certain embodiments, cell lines that can be used with the device include neuronal or cardiac cell lines that endogenously express ion channels, as well as cells that express cloned, exogenously introduced ion channels. An ‘excitable’ cell is one that can be fired, i.e., is one that generates an action potential. As such, an excitable cell typically has ion channels.

As used herein, the term “ion channel activity” means a change in state of an ion channel that allows it to alter its ion permeability. The terms “cell stimulation” or “cell excitation” mean the depolarization of a cell, such as may be achieved by destroying its membrane potential.

Activity in cells, as used herein, means the ‘firing’ of a cell through opening or closing of its ion channels. A drug may modulate activity of a cell by changing aspects of its ion channel function. “Cellular activity” means basic cell metabolism.

The terms “cell growth” and “cellular growth” mean the multiplication of metabolically active living cells.

The cellular activity to be measured by the present invention may be ion channel activity. Activity, such as ion channel activity of stimulated cells, is preferably measured through the use of one or more fluorescent dyes, added to the cells for detection purposes. When the cells have added fluorescent dye, the detection may, for example, detect a change in fluorescence over time.

Milieu

As used herein, the term “growth medium” is a composition, usually in liquid form, of the required compounds and nutritional energy source(s) suitable for promoting cell division when the cells are immersed in it. A growth medium is usually specific to a given cell type and has a composition known, or accessible to, one of ordinary skill in the art.

The surface of the silicon wafer is made suitable for cellular growth by applying a solution of poly-D-lysine in a buffer, preferably a borate buffer, optionally followed by a solution of laminin, for promoting cell growth. Optionally, also a silicon oxide layer is formed on the wafer, prior to applying the growth media.

Control System

As used herein, the term “controller” or “control circuit” mean electrical circuitry that includes at least one microprocessor programmed with software to control the operation of the device. The control circuitry preferably further comprises a first electrode, a second electrode, a voltage source and a microprocessor.

The control system may apply different sequences of pulses of both voltage and light to different locations on the assay plate.

The control system is coupled to the photodetection system and is used to control and analyze captured light, preferably from two distinct regions of the surface of a silicon wafer, one that is being stimulated through photoconductivity and one that is not. This is preferred for ordinary usage where one part of the surface is used as an experimental control. In a preferred embodiment, the stimulated region is a central region of the wafer and the annular region around the central region is unstimulated. In other embodiments, a control is provided by a wafer that is immersed in a solution that does not have any test compound.

Included in the control system is a microchip that has control of the light source and the electric field applied across the silicon wafer. The chip contains a microcontroller with firmware that enables the control of several input and output (I/O) ports (electronic circuits provide both digital and analog inputs and outputs, respectively). All analog inputs are converted to digital values by an analog to digital converter (ADC), and analog outputs are converted from digital values inside the microcontroller using digital to analog converters (DAC). The preferred A/D conversion technique is by the use of capacitor divider networks, and the DAC, by pulse-width modulation. A subset of the digital I/O (some microcontrollers have dedicated blocks to implement a serial interface) is used to implement a standard computer interface such as RS-232 or USB-1. This interface will be used to provide access to the microcontroller by a host computer. For a multiple of microcontrollers in a group of plate wells, a network or some other form of multiple-processor communication is established to enable a single host computer to access all microcontrollers in the plate wells. The host computer is used to initiate controlled operation of the microcontrollers in the plate wells; the microcontrollers, in turn, take local control of the experiments being conducted using the silicon wafer cell networks. An analog output port is connected to an on-chip variable regulated voltage circuit which powers the controlled voltage pulse source. There are many alternative ways of generating and controlling the pulse source, as would be understood by one of ordinary skill in the art. It is important to be able to control the type of signal that the pulse source generates (voltage, current, frequency, pattern, etc.), and variations applicable to different types of cells and classes of test compound, are within the capability of one of ordinary skill in the art.

The role of the control system includes, but is not limited to the following roles:

Activating the photoconductive stimulation light source, which in turn induces a photoconductive region in the silicon die.

Applying voltage pulses across the lower and upper electrodes which will cause the cells residing above the photoconductive region to be depolarized. A typical pulse duration is approximately 2 milliseconds. The frequency and length of time during which pulses are applied will depend on the particular test protocol. The amplitude of the pulse is determined by the control systems. It must be sufficient to cause depolarization of the cell, but not so large as to damage the cells.

Signalling the external drug delivery system to apply the drug under test to the appropriate wells.

Activating the fluorescence imagers at the appropriate times (as determined by the test protocol) and collect the required measurement data.

Method

The invention also comprises a method for stimulating cells with photoconductive stimulation in order to activate frequency dependent ion channels. This method is preferably carried out with standard optical imaging systems in conjunction with a suitably configured high throughput assay device. The device is used to determine the effect of drug compounds on ion channel activity.

Methods of the present invention may be used for stimulating neurons. Such methods may comprise the steps of: growing a cell line or native cells on a silicon wafer within wells on a plate; targeting the silicon wafer through photoconductivity and stimulating the targeted cells with an electric field; and reading a chemical's effect on the cell line or native cells indirectly through fluorescent dyes. The cell line or native cells may be placed on the substrate coating the silicon wafer using a pipette system. The plates may be placed in an incubator to support growth.

In these methods, the controller, which may be a microprocessor, controls when, in what pattern and towards which plate well the light-addressed electric field is applied. The voltage source may fire rapid sequences of voltage pulses.

### EXAMPLES

### Example 1

### Assay Apparatus

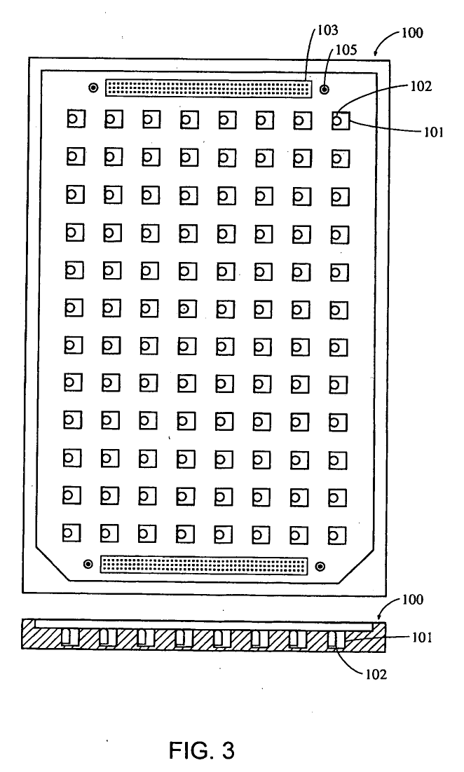

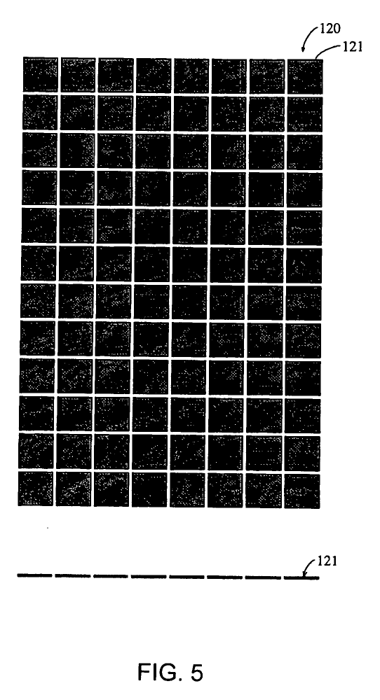

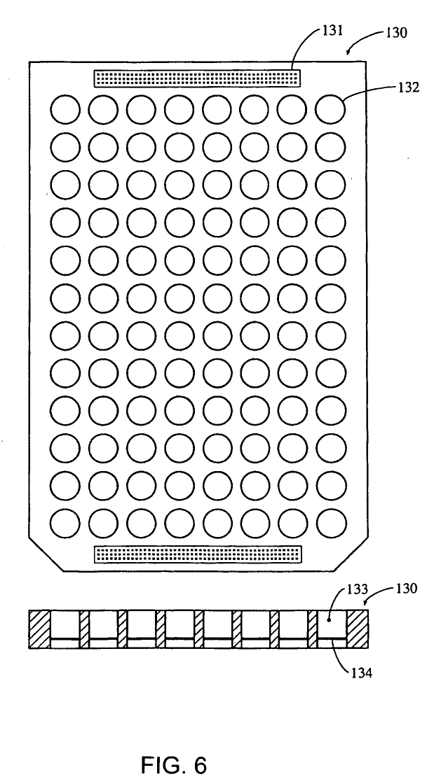

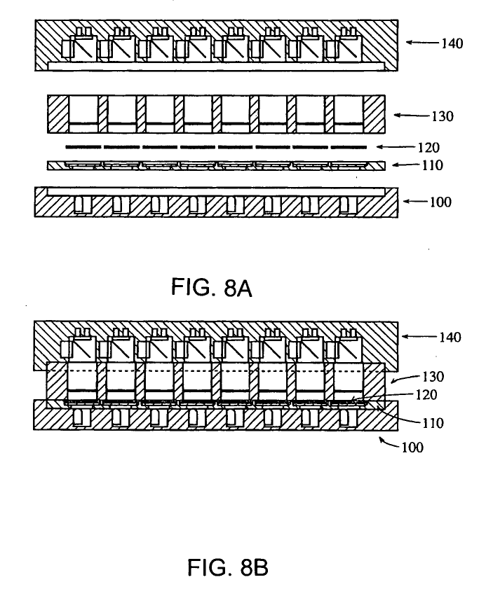

The components of a 96-well assay system, according to one embodiment of the invention, are shown in FIGS. 3-9. Although the example system contains 96 wells, the same approach could be applied to a system with a different number of wells, for example, 64, 100, 128, 256, or 512. In the representative embodiment (shown in cross-sectional view from the side in FIGS. 8A and 8B), the system is comprised of three major subassemblies: a bottom plate assembly 100 containing the light sources used for the photoconductive stimulation; an assembly having a middle plate 130 containing the wells, an array of silicon wafers 120, and upper and lower electrodes, and a carrier plate 100; and an upper plate assembly 140 containing the fluorescence excitation light source, dichroic mirror and filter assembly, and the fluorescence imagers. FIG. 6 shows an array of wells 132 disposed in middle plate 130. FIG. 5 shows a wafer assembly layer 120 having an array of individual wafers 121, each of which corresponds to a well 132.

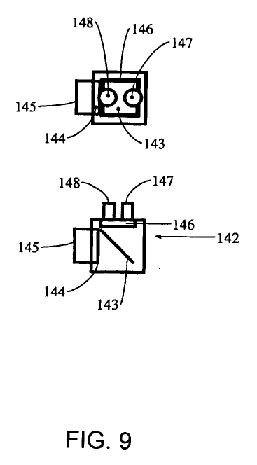

FIG. 3 shows an elevated view of the bottom plate assembly 100 which contains the light source 102 used to induce the photoconductive region in a silicon wafer (not shown) which is disposed above assembly 100. Each light source 102 is offset from a centerline (shown as a centerline parallel to the longer edge of the plate, in FIG. 3, but not limited to such an axis) of each respective well 132 (see FIG. 6) so as to allow each wafer to have a stimulated and un-stimulated region, as further described in connection with FIG. 9 hereinbelow. Each of the wells 132 has its own photoconductive light source 102 so as to allow all wells to be stimulated at the same time, or independently, as desired.

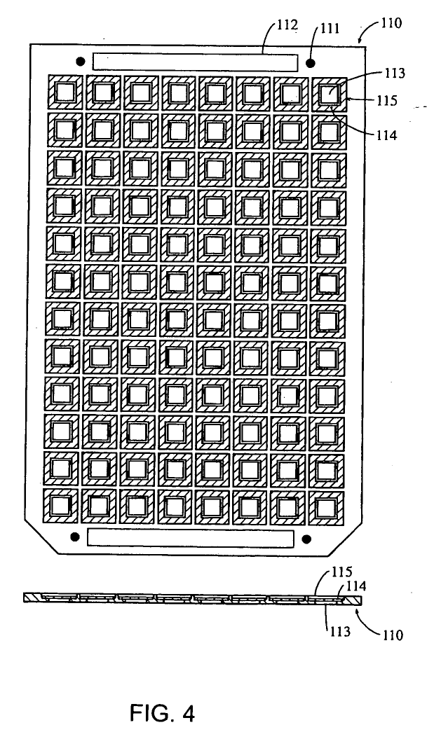

In between the bottom plate assembly 100 and the middle plate 130 are two sub-assemblies. Middle plate 130 contains the wells, is coupled to a wafer assembly, which in turn is coupled to a carrier plate 110, as shown in FIGS. 8A and 8B. FIG. 4 shows the carrier plate 110. The individual wafers 121 (not shown in FIG. 4) are placed in indentation 115 formed in the carrier plate 110. The bottom of each indentation contains a springy metallic contact 114 that makes contact with the underside of each silicon wafer 121. Each silicon wafer 121 has a transparent conductive oxide coating, in some cases indium tin oxide (ITO), on the bottom surface that allows the metallic contact 114 to make an electrical connection to the bottom surface of the wafer 121, yet be optically transparent to the light source 102. A hole 113 exists in the center region of each indentation 115 to allow the light from the source 102 to travel to the bottom surface of each wafer 121 unimpeded. During construction, the array of wafers 120 are situated in the carrier plate 110 and then the well plate 130 is attached to the carrier and wafer assembly using a ring of an adhesive, such as epoxy, around the outer edge of each wafer 121 to ensure that a watertight seal is created between the well plate 130 and each wafer 121.

The silicon wafers 121 are obtained by dicing (cutting) a silicon wafer having the characteristics as previously described herein, preferably single-side polished, and approximately 0.5 mm thick. Each wafer in the instant embodiment is about 8 mm by 8 mm. The size of the wafer is determined largely by the size of the wells 132. Each wafer is slightly larger than the diameter of the respective well 132 to allow sufficient area for the application of an adhesive, such as an epoxy, to glue the wafer to the middle plate assembly 130. A thin layer of oxide is preferably grown on the surface of the wafer prior to use. In some embodiments, this can be accomplished by ozonifying the wafers for approximately 15 minutes at room temperature. Each silicon wafer 121 may also comprise a user-applied growth substrate coated on the wafers. Such a substrate may comprise a chemical compound or cellular matrix that promotes cellular growth and survival on silicon.

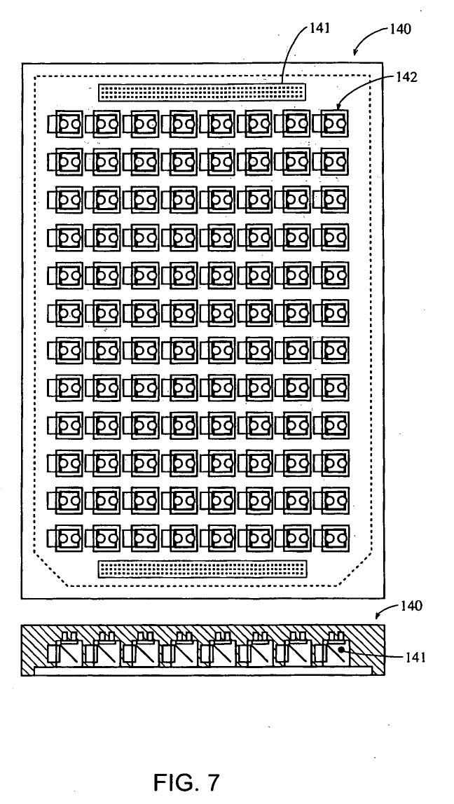

The upper plate assembly 140 shown in FIG. 7 contains the fluorescence excitation light source and associated filters and imager. Above each well 132 there is a subassembly 142 (shown in further detail in FIG. 9) containing an excitation light source 145, an excitation filter 144 which preferentially passes the desired wavelength of the light source 145, a dichroic mirror 143 which preferentially reflects light at the excitation wavelength but allows the fluorescence emission wavelength light to preferentially pass through to two imagers 148 and 147. An emission filter 146 in front of the imagers preferentially passes the fluorescence emission wavelength but blocks light at the excitation wavelength. For each well, two imagers are used in the upper plate 140 (FIG. 7). One imager 148 is aligned with the photoconductive stimulation light source 102 and measures the fluorescence of stimulated cells. The second imager 147 images a region of the cell culture that does not undergo photoconductive stimulation and thereby measures the fluorescence of cells in an unstimulated region of the wafer in order to provide a baseline reference for the measurements. Imager 147 is used to provide a reference level against which to compare the stimulated reading from imager 148.

Subassemblies 110, 120 (an array of individual wafers), and 130 are assembled into a composite component prior to use. To perform an assay, each individual well in the composite assembly is prepared with the desired cell culture and drug to be tested. The composite assembly is placed on top of assembly 100 and then assembly 140 is placed on top of subassembly 130.

For purposes of clarity, the various diagrams presented do not explicitly show the electrical connectivity needed for the purposes of powering, controlling, and measuring. However the base plate 100 contains, or is connected to, a microcontroller that performs all necessary local control and measurement functions. Control, power, and measurement signal are distributed to the middle and upper plates via the electrical connectors 103, 131, and 141 (FIGS. 3, 6, 7, respectively). The bottom electrode for all wells is maintained at the same potential, and electrical connectivity for this is provided via the connectors formed by 105 and 111 (FIGS. 3 and 4). The thinness of assembly 110 prevents a regular connector from being used, so the connector 105 (FIG. 3) is a spring loaded pogo pin and 111 (FIG. 4) is a metallic receptacle for the pogo pin. Plate 110 also contains holes 112 to allow connectors 105 and 131 to pass through.

As described previously, the bottom electrode 114 forms a mechanical and electrical contact with an indium tin oxide layer deposited on the bottom of the silicon wafer 121. The upper electrodes 134 are attached to the lower part of each well 132 in plate 130. An upper electrode may consist of a platinum wire submerged in the cell growth media present in each well during use. This media is electrically conductive and allows an electrical connection to be made between the upper electrode 134 and the upper surface of the silicon wafer 121.

### Example 2

### Photoconductive Stimulation of Human Embryo Kidney Cells

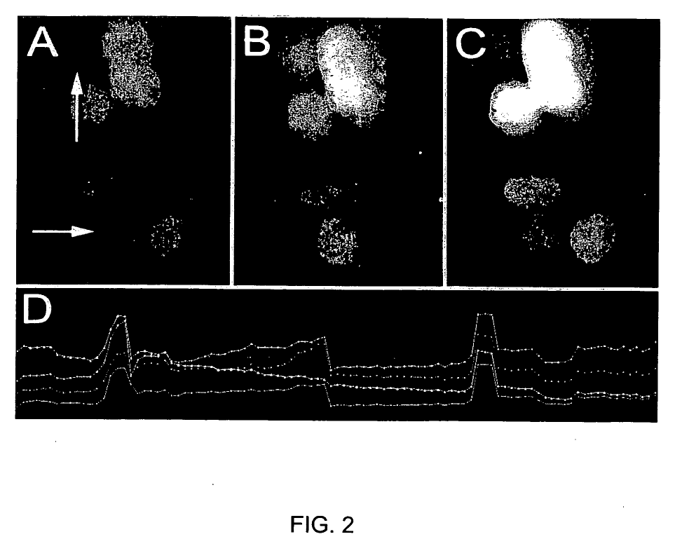

In FIG. 2, images A-C show Fluo4 calcium imaging of HEK cells which have been transfected with calcium channel subunits before (A) and after (B, C) depolarization with photoconductive stimulation. An increase in the level of calcium within the cells can be observed by the increase in fluorescence. Arrows identify cells that change. Panel (D) is a graph that demonstrates quantitative measurement of calcium indicator signal levels in a subset of cells. Depolarizations can be detected by peaks on the graph.

The foregoing description is intended to illustrate various aspects of the present invention. It is not intended that the examples presented herein limit the scope of the present invention. The invention now being fully described, it will be apparent to one of ordinary skill in the art that many changes and modifications can be made thereto without departing from the spirit or scope of the appended claims.

What is claimed:

1. A system for screening a test compound for activity in cells, the system comprising:
a silicon wafer having a surface suitable for cellular growth, and configured to be in contact with a growth medium, wherein the growth medium contains the test compound;
at least one cell in contact with the surface;
a light source configured to direct a light pulse to repetitively stimulate the at least one cell;
control circuitry connected to the silicon wafer and to the light source; and
a detector configured to detect the biochemical effects of the test compound on the at least one cell.

2. The system of claim 1, wherein the control circuitry comprises a first electrode, a second electrode, a voltage source, and a microprocessor.

3. The system of claim 1 wherein the light source is configured to direct the light pulse in a defined pattern at a defined wavelength.

4. The system of claim 2 wherein the voltage source is configured to direct a voltage pulse in a defined pattern in the surface of the silicon wafer.

5. The system of claim 4, wherein the voltage source is configured to provide a constant voltage.

6. The system of claim 3, wherein the light source is configured to direct a constant light beam.

7. The system of claim 1, wherein the at least one cell is attached to the surface.

8. The system of claim 1 wherein the detector detects fluorescence.

9. The system of claim 1 wherein the detector is a photodetector.

10. The system of claim 7, wherein the detector is a CCD.

11. The system of claim 1, further comprising a filter disposed between the surface and the detector.

12. The system of claim 11, further comprising a second light source configured to direct a pulse towards a portion of the silicon wafer that is not in contact with the at least one cell.

13. The system of claim 11, wherein the filter is configured to distinguish excitation light from the at least one cell, from emission light from the at least one cell.

14. The system of claim 11, wherein the filter comprises an optical filter and a dichroic mirror.

15. The system of claim 1 wherein the at least one cell expresses a voltage gated ion channel.

16. The system of claim 1 wherein the at least one cell is a neuron or a cardiac cell.

17. The system of claim 1 wherein in the at least one cell is selected from an excitable cell line that expresses an ion channel target.

18. The system of claim 1 wherein a fluorescent dye is added to the growth medium.

19. The system of claim 1 wherein the activity is firing the cell through opening and closing of an ion channel.

20. The system of claim 1, wherein the cells are stimulated for a period between 0.5 and 5 ms.

21. An array of systems according to claim 1, wherein each system of the array of systems is independently controllable.

22. An array of systems according to claim 21, wherein each of the systems is located in a well of an assay plate.

23. A method for screening a test compound for activity in cells, the method comprising:
providing a silicon wafer having a surface suitable for cell growth;
culturing at least one cell on the surface;
contacting the at least one cell with a test compound;
repetitively stimulating the at least one cell so as to excite the cell by directing a light pulse onto the at least one cell, wherein the light pulse is controlled by control circuitry; and
detecting a biochemical effect of the test compound on the at least one cell as a result of stimulated excitation of the cells.

24. The method of claim 23 wherein the activity is modulation of ion channel function.

25. The method of claim 23 wherein the cells are neurons or cardiac cells.

26. The method of claim 23 wherein the cells are from an excitable cell line expressing an ion channel target.

27. The method of claim 23 wherein a fluorescent dye is added to the growth medium.

28. The method of claim 23 wherein detecting detects a change in fluorescence.
